(19) United States
(12) Patent Application Publication
(10) Pub. No.: US 2022/0325277 A1
(43) Pub. Date: Oct. 13, 2022

(54) GENOMIC EDIT DETECTION AT THE SINGLE CELL LEVEL

(71) Applicant: Inscripta, Inc., Boulder, CO (US)

(72) Inventors: Burak Dura, Boulder, CO (US); Christine Bendzinkski, Boulder, CO (US); Kara Juneau, Boulder, CO (US)

(21) Appl. No.: 17/717,119

(22) Filed: Apr. 10, 2022

Related U.S. Application Data
(60) Provisional application No. 63/174,499, filed on Apr. 13, 2021.

(51) Int. Cl. C12N 15/10; C12Q 1/6806; C12Q 1/6874; C12Q 1/6855

(52) U.S. Cl. CPC C12N 15/1096; C12Q 1/6806; C12Q 1/6874; C12N 15/1093; C12Q 1/6855; C12N 15/1065; C12Q 2525/191

(57) ABSTRACT

Provided are methods and compositions for detecting genome editing events at the single cell level. The methods and compositions described herein utilize sequence-based methods with combinatorial barcoding to track the identity of single cells over single or multiple genome editing events.

### CROSS-REFERENCE TO RELATED APPLICATIONS

This application claims benefit of U.S. provisional patent application Ser. No. 63/174,499, filed Apr. 13, 2021, which is herein incorporated by reference in its entirety.

### FIELD OF THE INVENTION

The present disclosure relates to methods and compositions for detecting genome editing events at the single cell level, as well as devices and systems for performing these methods and using these compositions.

### BACKGROUND OF THE INVENTION

In the following discussion, certain articles and methods will be described for background and introductory purposes. Nothing contained herein is to be construed as an “admission” of prior art. Applicant expressly reserves the right to demonstrate, where appropriate, that the articles and methods referenced herein do not constitute prior art under the applicable statutory provisions.

In recent years, advances in next-generation sequencing (NGS) platforms have resulted in dramatically increased throughput capacity and significantly reduced sequencing costs. This progress has allowed for: rapid and affordable resequencing of large numbers of genomes; de novo genome sequencing of many diverse microbial, plant, and animal species; targeted resequencing studies, e.g., exome sequencing; and increasingly sensitive variant detection in human disease contexts, such as cancer. NGS techniques have further led to a shift from bulk sequencing to sequencing at the single cell level, thus facilitating genome-wide analysis of editing events at higher resolution.

Previous sequencing-based approaches to genomic edit screening involve DNA sequencing of pooled samples, wherein sequencing libraries are prepared from a combined pool of edited cells or colonies and single cell identities are not tracked. While cost-effective, such approaches lack single cell resolution, thereby limiting the ability to detect combinatorial or linked edits in a single cell or colony. With the shift of NGS techniques to single cell sequencing, more advanced genomic screening approaches with single cell resolution have been recently realized.

Current sequencing-based approaches to genomic edit screening at the single cell level, however, are not without limitations. For example, many single cell methods require compartmentalizing individual cells, which can be costly and can limit throughput and/or amplification of target nucleic acids. Where higher throughput is achieved, the sequencing platforms can only perform targeted sequencing with a limited number of edited loci, as compared to whole genome sequencing. The amplification techniques for single cell methods are also typically PCR-based, and thus, may suffer from exponential amplification biases that skew sequencing data uniformity. In certain cases, these methods can suffer from over 50% loss of target DNA.

Furthermore, current single cell RNA sequencing-based methods utilize the presence of guide RNAs as indicators for corresponding edits, thus relying on proxies to detect edits instead of direct detection at the genomic level. The accuracy of such an approach is therefore limited, as the presence of guide RNA does not guarantee the presence of a genomic edits. Even further, RNA sequencing-based methods are only applicable to cells with sufficient RNA content and are not suitable for cell types having low RNA expression, such as bacterial cells. Therefore, despite having transformative potential, current single cell sequencing methods are still limited with respect to throughput, uniformity, and cell compatibility.

Accordingly, there is a need in the art for improved methods and compositions for single cell sequencing to detect genome editing events. The present disclosure addresses this need.

### SUMMARY OF ILLUSTRATIVE EMBODIMENTS

This Summary is provided to introduce a selection of concepts in a simplified form that are further described below in the Detailed Description. This Summary is not intended to identify key or essential features of the claimed subject matter, nor is it intended to be used to limit the scope of the claimed subject matter. Other features, details, utilities, and advantages of the claimed subject matter will be apparent from the following written Detailed Description including those aspects illustrated in the accompanying drawings and defined in the appended claims.

The present disclosure relates to systems, methods, compositions, and devices for detecting genome editing events at the single cell level. The disclosure includes methods of using nucleic acid-guided editing and linear amplification of barcoded nucleic acids for single cell whole genome sequencing. Sequencing at the single cell level can provide a great deal of information regarding the efficiency of single or multiple editing events within a population of cells as compared to bulk sequencing. Furthermore, linear amplification of single cell genomic DNA avoids many limitations of other PCR-based amplification events, such as exponential amplification biases that skew sequencing data uniformity.

In some aspects, the single cell resolution of the methods described herein enables combinatorial and/or linked edit detection by tracking the identity of individual single cells.

In some aspects, detected genome editing events include, but are not limited to, single nucleotide changes, insertions, deletions, replacements, and/or modifications.

In some aspects, the disclosure provides a method of single cell whole genome pre-amplification in hydrogel beads. For example, single cells (e.g., prokaryotic or eukaryotic cells) can be loaded into hydrogel beads and lysed to release single cell genomic DNA, which is thereafter pre-amplified to increase single cell genomic mass available for preparation of single cell DNA libraries. The hydrogel beads provide compartmentalization or housing for the single cell genomes and facilitate handling/manipulation thereof in isolation without loss or mixing of genomic material.

In specific aspects, the pre-amplification of single cell whole genomes is achieved via multiple displacement amplification (MDA). In other aspects, pre-amplification is achieved via other methods, including PCR, loop mediated isothermal amplification, rolling circle amplification, and ligase chain reaction.

In some aspects, the disclosure provides a method of nucleic acid fragmentation using transposome complexes including a transposase subunit bound to a transposase recognition site sequence or “transposon.” Accordingly, in some aspects, the transposome complex can insert the transposon into a target nucleic acid via a process termed “tagmentation.”

In some aspects, the transposome complexes include a dimeric transposase having two subunits, and two non-contiguous transposon sequences. For example, each subunit of the dimeric transposase can be bound to one of the two non-contiguous transposon sequences, which may be the same or different. In certain other aspects, the transposome complexes include a dimeric transposase having two subunits, which are bound to a contiguous transposon sequence. In further aspects, the transposome complexes provided herein include a transposase subunit bound to two or more transposon sequences, which may or may not be linked to one another.

In specific aspects, the transposome complexes include a Tn5 transposase and a transposon having a Tn5 recognition site, e.g., a mosaic end sequence (ME sequence). In certain aspects, the transposome complexes include a MuA, Tn10, Tn7, Tn3, IS5, or similar transposase and/or recognition site.

In specific aspects, the transposons of the transposome complexes include a T7 RNA promoter sequence and primer sequence for sequencing library preparation, e.g., linear amplification via in vitro T7 transcription.

In some aspects, the transposons of the transposome complexes include a barcode sequence, also referred to as an index sequence or a tag sequence, the presence of which may be utilized to indicate a genome editing event. A barcode sequence can be any number of nucleotides in length, e.g., 1, 2, 3, 4, 5, 6, 7, 8, 9, 10, 11, 12, 13, 14, 15, 16, 17, 18, 19, 20, or more nucleotides.

In some aspects, sequencing libraries from single cells can be prepared utilizing unique barcode sequences for each unique single cell. For example, unique barcode sequences may be included in transposons to be inserted into nucleic acid sequences from single cells prior/subsequent to linear amplification thereof. In specific aspects, the unique barcode sequences for each unique single cell include nucleic acid sequences introduced at two or more separate barcoding events that do not require ligation processes, thus increasing genomic coverage and uniformity while reducing or eliminating ligation-related barcoding inefficiencies.

In some aspects, the transposons of the transposome complexes are hairpin oligonucleotides having complementary ME sequences that form a hairpin during annealing and facilitate self-priming of amplified products during reverse transcription. In other aspects, the transposons of the transposome complexes are linear oligonucleotides having two separate ME sequences that come together during annealing, thus requiring external priming of amplified products during reverse transcription.

In specific aspects, sequencing libraries are prepared utilizing in vitro T7 transcription (e.g., utilizing a T7 RNA polymerase with a T7 promoter) to linearly amplify tagmented genomic DNA sequences. Linear amplification may reduce or eliminate exponential amplification bias, thus facilitating improved uniformity of sequencing data with reduced error. In other aspects, linear amplification of the tagmented genomic DNA is achieved via other methods, including PCR, linked linear amplification, and linear extension with or without ligation.

In some aspects, upon transcription of tagmented genomic DNA sequences into amplified RNA copies, reverse transcription is performed to obtain single stranded cDNA copies, followed by second strand synthesis to convert the single stranded cDNA into double stranded molecules. In some aspects, second strand synthesis includes addition of a barcode sequence into the double stranded molecule, thus providing unique barcode combinations with previously inserted barcode sequences to facilitate unique barcode combinations for each unique single cell.

In some aspects, the double stranded cDNA molecules can be collected and/or purified prior to further analysis. In some aspects, the double stranded cDNA molecules can be sequenced using methods known to those of skill in the art. Upon sequencing, the sequences can be computationally analyzed to identify genome editing events at the single cell level.

The cells that can be sequenced using the methods of the present disclosure generally include mammalian and microbial cells. However, the methods described herein can be applied to any suitable eukaryotic or prokaryotic cells.

These aspects and other features and advantages of the invention are described below in more detail.

### BRIEF DESCRIPTION OF THE DRAWINGS

The foregoing and other features and advantages of the present invention will be more fully understood from the following detailed description of illustrative embodiments taken in conjunction with the accompanying drawings in which:

FIG. 1 depicts a flow diagram of an exemplary process for a sequencing-based approach to genomic edit screening in single cells, according to embodiments described herein.

FIG. 2 schematically depicts operations of the process of FIG. 1, according to embodiments described herein.

FIG. 3 schematically depicts an exemplary process for encapsulating single cells in gel beads, according to embodiments described herein.

FIGS. 4A-4B schematically depict an exemplary transposome complex and components thereof, according to embodiments described herein.

FIGS. 4C-4D schematically depict exemplary transposon oligonucleotides for tagmentation of genomic DNA, according to embodiments described herein.

FIG. 5A schematically depicts an exemplary process for nucleic acid gap extension of tagmented DNA, according to embodiments described herein.

FIG. 5B schematically depicts an exemplary process for linear amplification of tagmented DNA fragments, according to embodiments described herein.

FIG. 6A schematically depicts an exemplary process for reverse transcription of RNA transcripts into a cDNA library, according to embodiments described herein.

FIG. 6B schematically depicts an exemplary process for cDNA second strand synthesis, according to embodiments described herein.

FIG. 7 schematically depicts an exemplary process for ligation of sequencing adapters to a cDNA fragment, according to embodiments described herein.

It should be understood that the drawings are not necessarily to scale, and that like reference numbers refer to like features.

### The Invention in General

This disclosure is directed to nucleic acid sequencing for the detection of genome editing events. Generally, genome editing systems and tools require the utilization of assays to detect and provide information related to genome editing events in samples. However, current sequencing-based approaches to genomic edit screening have several limitations, including the inability to directly detect edits at the genomic level, the inability to track edits in individual single cells, limited throughput, and reduced uniformity of sequencing results. Furthermore, although applicable to mammalian cells, certain recent approaches, e.g., sci-Lianti, have not yet demonstrated compatibility with microbial cells. Thus, there is a growing need for sequencing-based assays that can detect genome editing events at the single cell level for both mammalian and microbial cells.

The present disclosure provides methods, compositions, and devices for sequencing-based detection of single and multiple genome editing events at the single cell level for both mammalian and microbial cells.

### DETAILED DESCRIPTION

All of the functionalities described in connection with one embodiment are intended to be applicable to the additional embodiments described herein except where expressly stated or where the feature or function is incompatible with the additional embodiments. For example, where a given feature or function is expressly described in connection with one embodiment but not expressly mentioned in connection with an alternative embodiment, it should be understood that the feature or function may be deployed, utilized, or implemented in connection with the alternative embodiment unless the feature or function is incompatible with the alternative embodiment.

The practice of the techniques described herein may employ, unless otherwise indicated, conventional techniques and descriptions of organic chemistry, polymer technology, molecular biology (including recombinant techniques), cell biology, biochemistry, biological emulsion generation, and sequencing technology, which are within the skill of those who practice in the art. Such conventional techniques include polymer array synthesis, hybridization and ligation of polynucleotides, and detection of hybridization using a label. Specific illustrations of suitable techniques can be had by reference to the examples herein. However, other equivalent conventional procedures can, of course, also be used. Such conventional techniques and descriptions can be found in standard laboratory manuals such as Green, et al., Eds. (1999), Genome Analysis: A Laboratory Manual Series (Vols. I-IV); Weiner, Gabriel, Stephens, Eds. (2007), Genetic Variation: A Laboratory Manual; Dieffenbach, Dveksler, Eds. (2003), PCR Primer: A Laboratory Manual; Bowtell and Sambrook (2003), DNA Microarrays: A Molecular Cloning Manual; Mount (2004), Bioinformatics: Sequence and Genome Analysis; Sambrook and Russell (2006), Condensed Protocols from Molecular Cloning: A Laboratory Manual; and Sambrook and Russell (2002), Molecular Cloning: A Laboratory Manual (all from Cold Spring Harbor Laboratory Press); Stryer, L. (1995) Biochemistry (4th Ed.) W. H. Freeman, New York N.Y.; Gait, “Oligonucleotide Synthesis: A Practical Approach” 1984, IRL Press, London; Nelson and Cox (2000), Lehninger, Principles of Biochemistry 3^{rd} Ed., W. H. Freeman Pub., New York, N.Y.; Berg et al. (2002) Biochemistry, 5^{th} Ed., W. H. Freeman Pub., New York, N.Y.; all of which are herein incorporated in their entirety by reference for all purposes. CRISPR-specific techniques can be found in, e.g., Genome Editing and Engineering from TALENs and CRISPRs to Molecular Surgery, Appasani and Church (2018); and CRISPR: Methods and Protocols, Lindgren and Charpentier (2015); both of which are herein incorporated in their entirety by reference for all purposes.

Note that as used herein and in the appended claims, the singular forms “a,” “an,” and “the” include plural referents unless the context clearly dictates otherwise. Thus, for example, reference to “an oligonucleotide” refers to one or more oligonucleotides, and reference to “an automated system” includes reference to equivalent steps and methods for use with the system known to those skilled in the art, and so forth. Additionally, it is to be understood that terms such as “left,” “right,” “top,” “bottom,” “front,” “rear,” “side,” “height,” “length,” “width,” “upper,” “lower,” “interior,” “exterior,” “inner,” “outer” that may be used herein merely describe points of reference and do not necessarily limit embodiments of the present disclosure to any particular orientation or configuration. Furthermore, terms such as “first,” “second,” “third,” etc., merely identify one of a number of portions, components, steps, operations, functions, and/or points of reference as disclosed herein, and likewise do not necessarily limit embodiments of the present disclosure to any particular configuration or orientation.

Unless defined otherwise, all technical and scientific terms used herein have the same meaning as commonly understood by one of ordinary skill in the art to which this invention belongs. All publications mentioned herein are incorporated by reference for the purpose of describing and disclosing devices, methods and cell populations that may be used in connection with the presently described invention.

Where a range of values is provided, it is understood that each intervening value, between the upper and lower limit of that range and any other stated or intervening value in that stated range is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

In the following description, numerous specific details are set forth to provide a more thorough understanding of the present invention. However, it will be apparent to one of ordinary skill in the art that the present invention may be practiced without one or more of these specific details. In other instances, well-known features and procedures well known to those skilled in the art have not been described in order to avoid obscuring the invention.

An “adapter” or “adapter sequence” is a polynucleotide or oligonucleotide which can be ligated to a nucleic acid.

As used herein, the terms “amplify” or “amplification” and their derivatives, refer to any operation or process whereby at least a portion of a nucleic acid molecule is replicated or copied into at least one additional nucleic acid molecule. The additional nucleic acid molecule may include a sequence that is substantially identical or substantially complementary to at least a portion of the template nucleic acid molecule. The template nucleic acid molecule can be single-stranded or double-stranded, and the additional nucleic acid molecule can be independently single-stranded or double-stranded. Amplification may include linear or exponential replication of a nucleic acid molecule. In certain embodiments, amplification can be achieved using isothermal conditions; in other embodiments, amplification may include thermocycling. In certain embodiments, the amplification is a multiplex amplification and includes the simultaneous amplification of a plurality of target sequences in a single reaction or process. In certain embodiments, “amplification” includes amplification of at least a portion of DNA and RNA based nucleic acids. The amplification reaction(s) can include any of the amplification processes known to those of ordinary skill in the art. In certain embodiments, the amplification reaction(s) includes methods such as polymerase chain reaction (PCR), ligase chain reaction (LCR), or other methods.

The term “complementary” as used herein refers to Watson-Crick base pairing between nucleotides and specifically refers to nucleotides hydrogen bonded to one another with thymine or uracil residues linked to adenine residues by two hydrogen bonds and cytosine and guanine residues linked by three hydrogen bonds. In general, a nucleic acid includes a nucleotide sequence described as having a “percent complementarity” or “percent homology” to a specified second nucleotide sequence. For example, a nucleotide sequence may have 80%, 90%, or 100% complementarity to a specified second nucleotide sequence, indicating that 8 of 10, 9 of 10 or 10 of 10 nucleotides of a sequence are complementary to the specified second nucleotide sequence. For instance, the nucleotide sequence 3′-TCGA-5′ is 100% complementary to the nucleotide sequence 5′-AGCT-3′; and the nucleotide sequence 3′-TCGA-5′ is 100% complementary to a region of the nucleotide sequence 5′-TTAGCTGG-3′.

The term DNA “control sequences” refers collectively to promoter sequences, polyadenylation signals, transcription termination sequences, upstream regulatory domains, origins of replication, internal ribosome entry sites, nuclear localization sequences, enhancers, and the like, which collectively provide for the replication, transcription and translation of a coding sequence in a recipient cell. Not all of these types of control sequences need to be present so long as a selected coding sequence is capable of being replicated, transcribed and—for some components—translated in an appropriate host cell.

The term “heterologous” refers to the relationship between two or more nucleic acids or protein sequences from different sources, or the relationship between a protein (or nucleic acid) and a host cell from different sources. For example, if the combination of a nucleic acid and a host cell is usually not naturally occurring, the nucleic acid is heterologous to the host cell. A particular sequence is “heterologous” to the cell or organism into which it is inserted.

“Homology” or “identity” or “similarity” refers to sequence similarity between two peptides or, more often in the context of the present disclosure, between two nucleic acid molecules. The term “homologous region” or “homology arm” refers to a region on the donor DNA with a certain degree of homology with the target genomic DNA sequence. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences.

“Operably linked” refers to an arrangement of elements where the components so described are configured so as to perform their usual function. Thus, control sequences operably linked to a coding sequence are capable of effecting the transcription, and in some cases, the translation, of a coding sequence. The control sequences need not be contiguous with the coding sequence so long as they function to direct the expression of the coding sequence. Thus, for example, intervening untranslated yet transcribed sequences can be present between a promoter sequence and the coding sequence and the promoter sequence can still be considered “operably linked” to the coding sequence. In fact, such sequences need not be located on the same contiguous DNA molecule (i.e. chromosome) and may still have interactions resulting in altered regulation.

A “primer” or “primer sequence” is a nucleic acid sequence that can hybridize to a target sequence and function as a starting point for nucleic acid synthesis. Generally, primers act as substrates onto which nucleotides can be polymerized by a polymerase or to which a nucleic acid sequence, e.g., a barcode sequence, can be ligated. In certain examples, the primer itself may be incorporated into the synthesized nucleic acid sequence. In certain examples, the primer is a single stranded polynucleotide or oligonucleotide.

A “promoter” or “promoter sequence” is a DNA regulatory region capable of binding RNA polymerase and initiating transcription of a polynucleotide or polypeptide coding sequence such as messenger RNA, ribosomal RNA, small nuclear or nucleolar RNA, guide RNA, or any kind of RNA. Promoters may be constitutive or inducible. A “pol II promoter” is a regulatory sequence that is bound by RNA polymerase II to catalyze the transcription of DNA.

As used herein, the term “selectable marker” refers to a gene introduced into a cell, which confers a trait suitable for artificial selection. General use selectable markers are well-known to those of ordinary skill in the art. Drug selectable markers such as ampicillin/carbenicillin, kanamycin, chloramphenicol, erythromycin, tetracycline, gentamicin, bleomycin, streptomycin, puromycin, hygromycin, blasticidin, and G418 may be employed. In other embodiments, selectable markers include, but are not limited to human nerve growth factor receptor (detected with a MAb, such as described in U.S. Pat. No. 6,365,373); truncated human growth factor receptor (detected with MAb); mutant human dihydrofolate reductase (DHFR; fluorescent MTX substrate available); secreted alkaline phosphatase (SEAP; fluorescent substrate available); human thymidylate synthase (TS; confers resistance to anti-cancer agent fluorodeoxyuridine); human glutathione S-transferase alpha (GSTA1; conjugates glutathione to the stem cell selective alkylator busulfan; chemoprotective selectable marker in CD34+cells); CD24 cell surface antigen in hematopoietic stem cells; human CAD gene to confer resistance to N-phosphonacetyl-L-aspartate (PALA); human multi-drug resistance-1 (MDR-1; P-glycoprotein surface protein selectable by increased drug resistance or enriched by FACS); human CD25 (IL-2a; detectable by Mab-FITC); Methylguanine-DNA methyltransferase (MGMT; selectable by carmustine); rhamnose; and Cytidine deaminase (CD; selectable by Ara-C). “Selective medium” as used herein refers to cell growth medium to which has been added a chemical compound or biological moiety that selects for or against selectable markers.

The terms “target genomic DNA sequence”, “target sequence”, or “genomic target locus” refer to any locus in vitro or in vivo, or in a nucleic acid (e.g., genome or episome) of a cell or population of cells, in which sequencing is desired. The target sequence can be a genomic locus or extrachromosomal locus.

A “vector” is any of a variety of nucleic acids that comprise a desired sequence or sequences to be delivered to and/or expressed in a cell. Vectors are typically composed of DNA, although RNA vectors are also available. Vectors include, but are not limited to, plasmids, fosmids, phagemids, virus genomes, BACs, YACs, PACs, synthetic chromosomes, and the like.

The present disclosure provides methods and compositions for a sequencing-based approach to detecting genome editing events at the single cell level. Current sequencing-based approaches to genomic edit screening suffer from several limitations, including low resolution, low throughput, reduced data uniformity, and high associated costs. Furthermore, current RNA sequencing-based techniques rely on proxies for detection of genomic edits, thus having reduced accuracy, and are only compatible with cells having sufficient RNA content (e.g., mammalian cells).

The methods and compositions disclosed herein address the limitations of existing sequencing-based genomic edit screening approaches by: providing edit detection directly at the genomic level instead of relying on proxies or other indirect approaches in which edits are inferred; providing sequencing coverage of whole genomes rather than limited numbers of loci as in targeted sequencing, which enables application of the methods and compositions herein to high plexity library screens (e.g., 100-plex, 1,000-plex, or more); enabling processing of 10,000 single cells or more, thus improving through put and reducing library preparation costs per cell; providing single cell resolution to enable combinatorial or linked edit detection by tracking the identities of individual cells; and providing improved compatibility with more types of cells, including both mammalian and microbial cells.

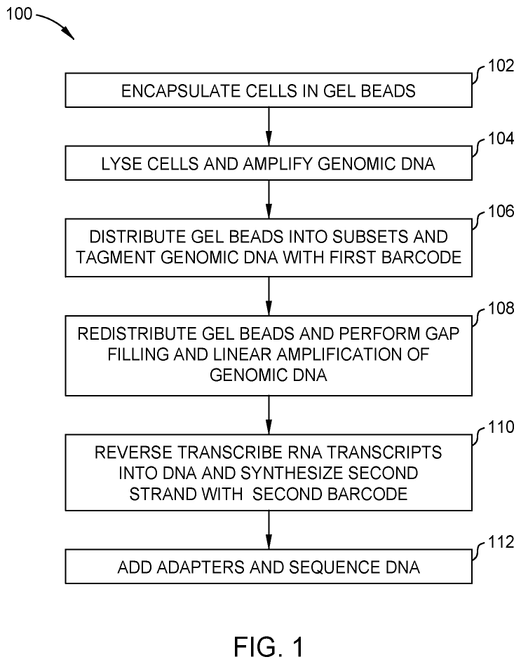

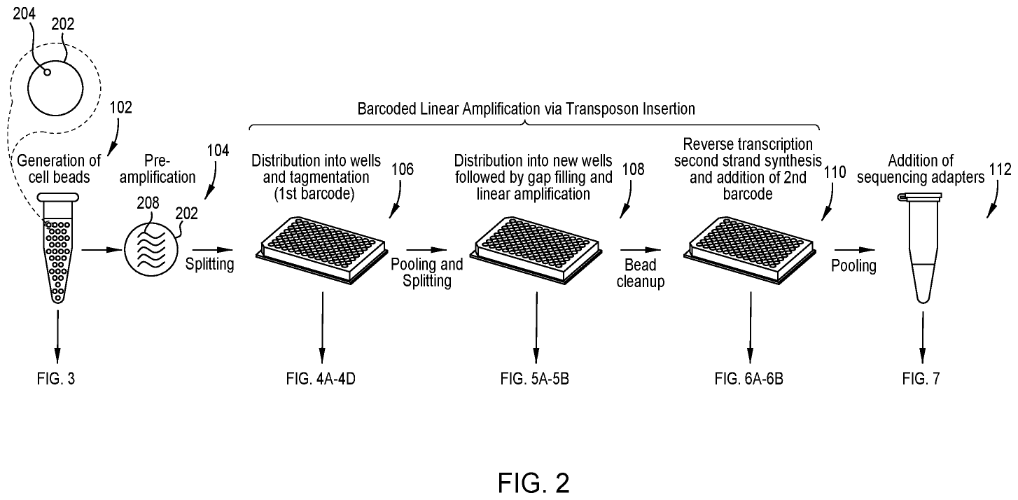

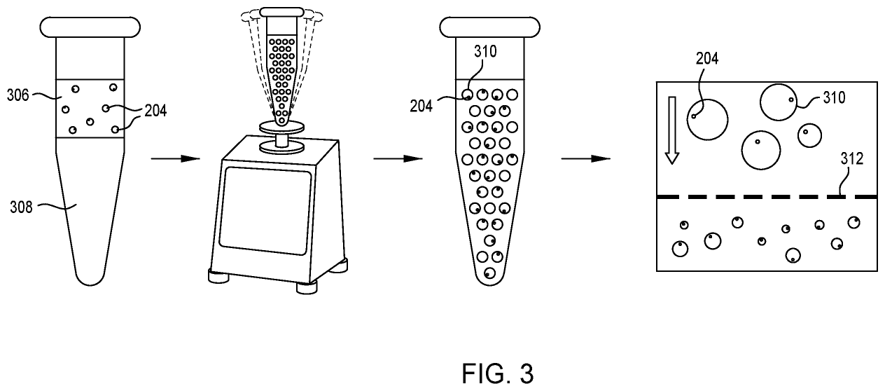

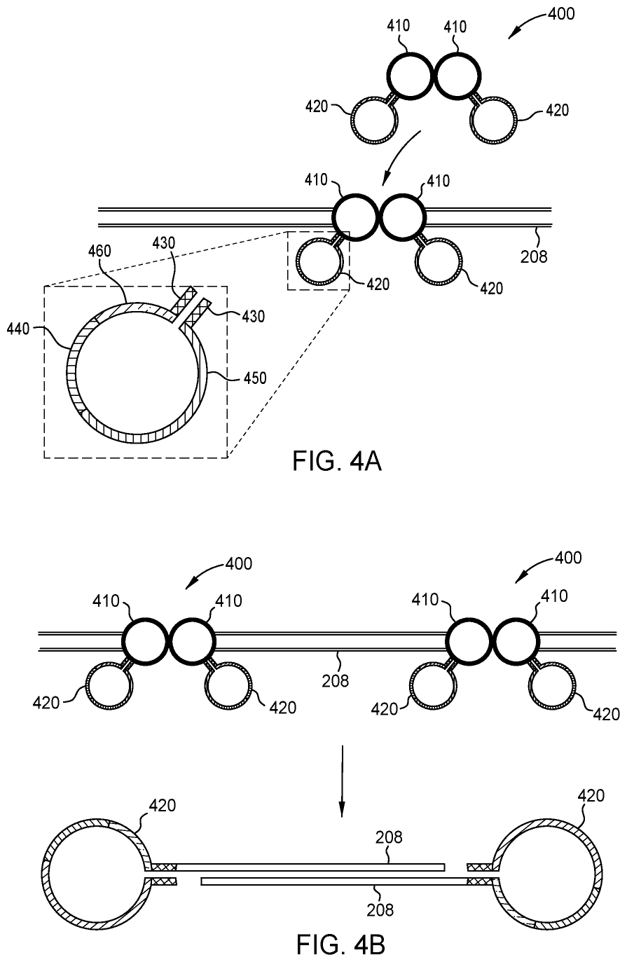

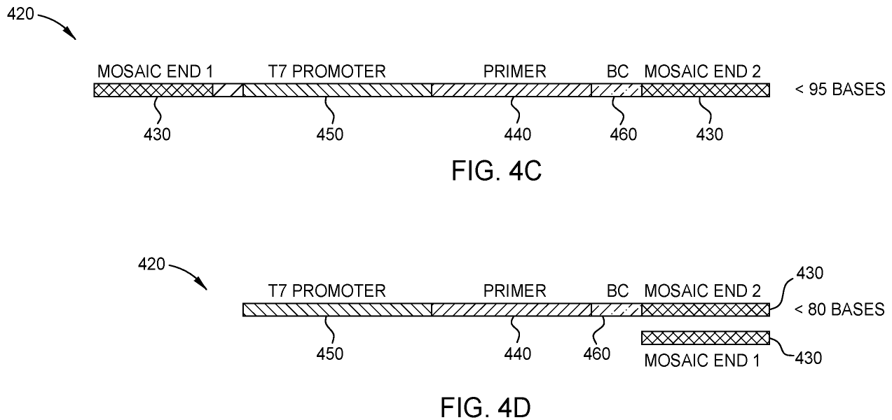

The methods and compositions disclosed herein include linear amplification reactions of tagmented genomic DNA to prepare combinatorial barcoded DNA sequences for sequencing-based identification and tracking of genome editing events in individual single cells. FIG. 1 depicts a flow diagram of an exemplary process 100 for a sequencing-based approach to genomic edit screening, according to embodiments described herein. FIG. 2, FIG. 3, FIGS. 4A-4D, FIGS. 5A-5B, FIGS. 6A-6B, and FIG. 7 schematically depict certain operations and/or elements of the process 100 of FIG. 1, according to embodiments. Therefore, FIG. 1 and FIGS. 2-7 are herein described together, where appropriate, for clarity.

### Single Cell Genomic Sample Preparation

### Cell Encapsulation in Gel Beads

The process 100 generally begins at operation 102, wherein single cells 204 are first captured and entrapped in gel beads 202 or similar microcarriers. The gel beads 202 are formed such that they can carry a payload of, e.g., one or more cells 204, single- or double-stranded DNA, and/or single-stranded RNA, which may be effectively encapsulated within the gel beads 202 without any substantial loss of genomic material. However, pores in each gel bead “mesh” allow cell lysis reagents, as well as nucleic acid amplification reagents, to penetrate the gel bead 202. Thus, the gel beads 202 provide single cell genome isolation (e.g., partitioning) while also facilitating cell lysis and whole genome of amplification of entrapped cells, and further enable more efficient handling as compared to the utilization of pre-existing cellular structures for genome partitioning.

The gel matrix forming the gel beads 202 typically comprises at least one polymer and a linker. The gel beads 202 may be porous, non-porous, solid, semi-solid, and/or semi-fluidic. In certain embodiments, the gel beads 202 are degradable, dissolvable, or disruptable. The gel beads 202 may be hydrogel beads, formed from molecular precursors such as a polymeric or monomeric species. The gel beads 202 may be of uniform size or heterogeneous size. In certain embodiments, the diameter of each gel bead 202 is at least about 1 micrometer (μm), 5 μm, 10 μm, 20 μm, 25 μm, 30 μm, 35 μm, 40 μm, 45 μm, 50 μm, 55 μm, 60 μm, 65 μm, 70 μm, 75 μm, 80 μm, 85 μm, 90 μm, 95 μm, 100 μm, 150 μm, 200 μm, 250 μm, 300 μm, 400 μm, 500 μm, 1 mm, or greater. Typically, in the present methods, the gel beads 202 are provided as a population or plurality of gel beads having a relatively monodisperse size distribution as it is desirable to provide relatively consistent amounts of reagents within the gel beads 202.

Gel beads 202 for use herein contain molecular precursors (e.g., monomers or polymers) which form a polymer network via polymerization of the molecular precursors. In certain embodiments, a precursor may be an already polymerized species capable of undergoing further polymerization via, for example, a chemical cross-linkage. For example, a precursor may comprise one or more of an acrylamide or a methacrylamide monomer, oligomer, or polymer (e.g., polyacrylamide). In some cases, the gel beads 202 may comprise pre-polymers, which are oligomers capable of further polymerization; for example, polyurethane beads may be prepared using prepolymers. Alternatively, the gel beads 202 may contain individual polymers that may be further polymerized together. In certain embodiments, the gel beads 202 may be generated via polymerization of different precursors, such that they comprise mixed polymers, co-polymers, and/or block co-polymers. In certain embodiments, the gel beads 202 may comprise covalent or ionic bonds between polymeric precursors (e.g., monomers, oligomers, linear polymers) and other entities. In some aspects, the covalent bonds can be carbon-carbon bonds or thioether bonds. In the present methods, cross-linking preferably is reversible, which allows for the polymer to linearize or dissociate under appropriate conditions. In some aspects of the present methods, reversible cross-linking may also allow for reversible attachment of a material bound to the surface of a gel bead 202.

In some aspects, disulfide linkages can be formed between molecular precursor units (e.g., monomers, oligomers, or linear polymers) incorporated into each of the gel beads 202. For example, cystamine and modified cystamines are organic agents comprising a disulfide bond that may be used as a crosslinker agent between individual monomeric or polymeric precursors of a gel bead 202. Polyacrylamide may be polymerized in the presence of cystamine or a species comprising cystamine to generate polyacrylamide gel beads comprising disulfide linkages; that is, chemically degradable beads comprising chemically-reducible cross-linkers. The disulfide linkages permit the bead to be degraded (or dissolved) upon exposure of the bead to a reducing agent.

In certain embodiments, such as schematized in FIG. 3, single cells 204 are encapsulated in the gel beads 202 by vortex emulsification. For example, in embodiments where the gel beads 202 are hydrogel beads, a hydrogel solution or aqueous phase 306, e.g., polyacrylamide with ammonium persulfate, may be mixed with single cells 204 and added to an oil phase 308, e.g., HFE-7500 fluorinated oil supplemented with 1% (vol/vol) TEMED. The combined aqueous and oil phases 306, 308 may then be vortexed to generate droplets 310 of hydrogel solution with single cells 204 trapped therein, and thereafter incubated for a predetermined incubation time to polymerize hydrogels within the droplets 310 into hydrogel beads 202.

The size of the hydrogel beads 202 may be adjusted by passing the droplets 310 through one or more strainers 312 prior to polymerization. For example, the droplets 310 may be exposed to 1, 2, 3, 4, 5, 6, 7, 8, 9, 10, or more passes through one or more strainers 312, which may include a filter, mesh, or other extrusion device having a pore size of about 20 μm, 30 μm, 40 μm, 50 μm, 60 μm, 70 μm, 80 μm, 90 μm, 100 μm, or greater. In certain embodiments, the droplets 310 are passed through at least two strainers 312 having different pore sizes to obtain a desired bead size.

Furthermore, the number of singly loaded hydrogel beads 202 may be adjusted by modifying the cell concentration of the aqueous phase 306. Cell encapsulation in the hydrogel beads 202 follows a Poisson distribution; thus, by adjusting the concentration of cells 204 mixed with the aqueous phase 306, the fraction or percentage of hydrogel beads 202 loaded with single cells 204 can be tuned. In operation, many beads 202 formed by the methods herein are left empty to prevent encapsulating more than one cell 204 per bead 202, thereby ensuring single cell resolution.

### Cell Lysis and Whole Genome Amplification in Gel Beads

At operation 104, the single cells 204 are lysed and the genomic DNA 208 therefrom is amplified. In certain embodiments, the population of gel beads 202 is exposed, in bulk, to lysis conditions sufficient to lyse the cells contained within the gel beads 202. In some embodiments, the polymerized gel beads 202 are permeable to reagents and the lysis conditions at operation 104 include exposing the polymerized gel beads 202 to one or more lytic enzymes. The lytic enzyme(s) may include any suitable lytic enzyme(s) known to those of ordinary skill in the art and may depend on the type of cells 204 utilized. Examples of suitable lytic enzymes include lysozymes, nucleases, and proteases (e.g., proteinase K). In certain embodiments, exposure of the gel beads 202 to the lytic enzymes may include incubating the gel beads 202 with the lytic enzymes for a suitable amount of time and at a suitable temperature sufficient to lyse the cells 204.

In some embodiments, the lysis conditions at operation 104 include exposing the already polymerized gel beads 202 to one or more detergents to solubilize cellular material of the cells 204 therein. For example, the detergents may solubilize cell membrane lipids that are released upon cell lysis. Suitable detergents for use with the methods described herein include those well known in the art. For example, in certain embodiments, the lysis detergents may include one or more of sodium dodecyl sulfate (SDS), SDS C12, SDS lauryl, 3-((3-cholamidopropyl) dimethylammonio)-1-propanesulfonate (CHAPS), NP-40, Triton X-100, Triton X-114, TWEEN-20, TWEEN-80, and the like. In certain embodiments, the gel beads 202 are exposed to two or more different detergents simultaneously or in successive separate steps. In certain embodiments, operation 104 includes exposing the gel beads 202 to one or more detergents and one or more lytic enzymes in series.

In further embodiments, lysis conditions at operation 104 include exposing the gel beads 202 to heat (e.g., thermal lysis), one or more freeze-thaw cycles, an alkaline solution (e.g., KOH of NaOH, for alkaline lysis), or any lysis methods and/or reagents known to those of ordinary skill in the art.

In certain embodiments herein, lysis reagents, nucleic acid amplification reagents, and/or the like may be encapsulated in the gel beads 202 during gel bead generation (e.g., during polymerization of precursors) and the gel beads 202 are not permeable to the reagents. In some aspects, the encapsulation of reagents and/or the addition of reagents after gel bead polymerization is controlled by the polymer network density of the gel beads 202. The porosity of gel beads 202 can be controlled by adjusting the polymer concentration or degree of crosslinking, effectively creating a tunable molecular cut-off size for transport through the gel. The porosity can then be adjusted to physically retain large molecules of interest while allowing smaller molecules or buffers to be freely exchanged. (See, e.g., Rehmann, et al., Biomacromolecules, 18(10):3131-42 (2017); Goodrich, et al., Nat. Communications, 9:4348 (2018); and Tsuji, et al., Gels, doi: 103390/gel4020050 (2018).). Alternatively, the polymer network may be chemically modified to conjugate specifically with a target molecule for retention. For example, in certain embodiments, one or more oligonucleotides and/or primers may be specifically conjugated to the polymer network within the gel beads 202 to capture and/or prime specific sequences of genomic DNA 208 for downstream events. As described herein infra, encapsulated reagents and molecules may be released from a gel bead 202 upon degradation of the gel bead.

Upon lysis of the cells 204, the genomic material thereof, e.g., genomic DNA 208, in the form of large molecular weight macromolecules, remains trapped within the polymerized gel beads 202. Accordingly, genomic DNA 208 from one cell 204 does not mix with genomic DNA 208 of another cell 204, thus efficiently compartmentalizing or partitioning the genomic material of individual single cells into respective gel beads 202. In certain embodiments, due to the porosity of the gel beads 202, smaller sized molecules, such as lysis and amplification reagents, may freely enter or exit the gel beads 202 while genomic material remains immobilized. In such embodiments, the trapped genomic material can be purified in between lysis and amplification. For example, the population of gel beads 202 may be exposed to one or more washing steps whereby the gel beads 202 are contacted with one or more washing buffers, simultaneously or in series, to remove any reagents or species therein that may inhibit downstream molecular biology reactions, e.g., amplification. The washing buffer(s) may include any suitable washing buffer(s) known to those of ordinary skill in the art and may depend on the type of lysis reagents utilized.

In order to increase the amount of single cell genomic mass available for downstream preparation of single cell DNA libraries, the genomic DNA 208 in each gel bead 202 is amplified after lysis by suitable amplification techniques. Note that amplification at operation 104 may also be referred to as “pre-amplification,” and the gel beads 202, now having amplified amounts of genomic DNA 208 therein, may be referred to as “amplified genome beads.” In certain embodiments, genomic DNA 208 is amplified via whole genome amplification (WGA) methods. Examples of suitable WGA methods for operation 104 include multiple displacement amplification (MDA), strand displacement amplification (SDA), PCR-based methods such as degenerate oligonucleotide PCR (DOP-PCR) and primer extension pre-amplification (PEP), exclusion amplification (ExAmp), multiple annealing and looping based amplification cycles (MALBAC), combinations thereof, and other methods known to those skilled in the art, which may be achieved utilizing commercially available kits. Isothermal amplification methods such as MDA may be used with, for example, strand-displacing phi29 polymerase or B st DNA polymerase for random primer amplification of the genomic DNA. Such polymerases have high processivity and strand displacing activity, the polymerases to produce large and highly branched fragments that are 10-20 kb in length. Alternatively, smaller fragments may be produced under isothermal conditions using polymerases having low processivity and strand-displacing activity, such as Klenow polymerase.

In certain embodiments, amplification at operation 104 is achieved via targeted amplification (i.e., “targeted pre-amplification”) approaches instead of WGA, wherein specific loci are amplified using one or more targeting primers and isothermal or PCR amplification, (e.g., multiplex PCR). However, it is to be understood that the present disclosure contemplates different amplification methods for amplifying genomic DNA of the single cells 204 and is not limited to any particular amplification method.

### Single Cell Dna Sequencing Library Preparation Through Split-Pool Barcoding

### Distribution of Gel Beads and Tagmentation

At operation 106, after pre-amplification, the gel beads 202 are distributed into subsets and the genomic DNA 208 therein is tagmented. Generally, distribution may include multiple distribution steps wherein a population or subpopulation of gel beads 202 is split and pooled into subsets separately contained within, e.g., individual wells of one or more multi-well plates, before tagmentation and insertion of barcode sequences. In certain embodiments, the number of splitting/pooling steps and/or number of subsets depends on the number of different barcode sequences to be added to the genomic DNA 208. For example, the number of splitting and/or pooling steps may be based, in part, on the desire to reduce the amount of genomic DNA 208 having the same barcode sequences inserted therein.

In certain embodiments, the number of subsets depends on a plate format being utilized and the number of wells or compartments thereof. For example, the number of subsets may range between 2 and 96 subsets, or multiples thereof, when a 96-well plate is used, or between 2 and 384 subsets, or multiples thereof, when a 384-well plate is used. Generally, the number of gel beads 202 distributed into each subset is at least 1. In certain embodiments, the number of gel beads 202 distributed into each subset is between about 10 and about 100, with a targeted number of cells between about 10 and about 150 per subset. For example, in embodiments where a 96-well plate is used, each well may contain an average of about 10 cells. In embodiments where a 384-well plate is used, each well may contain between about 10 and about 150 cells, with an average of about 50 cells. Methods for splitting and pooling gel beads 202 into subsets are known to those of ordinary skill in the art and are routine.

After distribution, the genomic DNA 208 in each subset is tagmented, i.e., fragmented and indexed (“tagged”) for downstream sequencing. In certain embodiments, tagmentation of genomic DNA 208 is achieved via utilization of a transposition system. For example, the distributed gel beads 202 may be exposed to a transposome complex, such as the exemplary transposome complex 400 depicted in FIGS. 4A-4B. The transposome complex 400 includes at least one subunit 410 of a transposase enzyme bound to at least one transposon 420, e.g., a user-defined oligonucleotide sequence, having a transposase recognition site. As shown in FIGS. 4A, in certain embodiments, the transposome complex 400 includes a dimeric transposase enzyme having two subunits 410 and two non-contiguous transposons 420. For example, each subunit 410 of the dimeric transposase can be bound to one of the two distinct transposons 420, which may be the same or different. In certain other embodiments, the transposome complex 400 includes two transposase enzyme subunits 410 bound to a single contiguous transposon 420, or two transposase enzyme subunits 410 bound to two or more transposons 420, which may or may not be linked to one another. In further embodiments, the 5′ end of one or both strands of the transposon 420 may be phosphorylated.

The transposase enzyme subunits 410 of the transposome complex 400 facilitate fragmentation of the target genomic DNA 208 and covalent attachment of the 3′ ends of the transposons 420 to the 5′ ends of the fragmented genomic DNA 208, schematically shown in FIG. 4B. In certain embodiments, the transposome complex 400 includes a hyperactive Tn5 transposase enzyme and a Tn5-type transposase recognition site. However, the transposition system described herein can include any of transposases known to those of ordinary skill in the art. For example, the transposome complex 400 may include a MuA, Tn10, Tn7, Tn3, IS5, or similar transposase enzyme and/or transposase recognition site.

FIGS. 4C-4D illustrate exemplary designs of the transposon 420, according to embodiments described herein. Generally, the transposon 420 can be any suitable number of nucleotides in length, such as 50, 60, 70, 80, 90, 100, 110, 120, 130, 140, 150, 160, 170, 180, 190, 200, or more nucleotides in length. Each transposon design typically includes one or more transposase recognition sites to facilitate binding with the transposase enzyme subunit(s) 410. For example, the transposon 420 may include a first transposase recognition site and a second transposase recognition site in the form of mosaic ends 430 disposed at the 3′ and/or 5′ ends thereof. In certain embodiments, as shown in FIG. 4C, the mosaic ends 430 are complimentary mosaic ends that form a hairpin structure during annealing thereof, enabling self-priming of amplified products during downstream amplification reactions. Accordingly, the transposon 420 in FIG. 4C may be referred to as a “hairpin oligonucleotide.” In certain embodiments, as shown in FIG. 4D, the mosaic ends 430 are separate mosaic ends that come together upon annealing, thus requiring external priming during downstream amplification reactions. Accordingly, the transposon 420 in FIG. 4D may be referred to as a “linear oligonucleotide.”

The transposon 420 may also include one or more optional sequences used by linear amplification mediators or regulators. For example, as shown in both FIG. 4C and FIG. 4D, the transposon 420 may include a primer sequence 440 and/or a looped promoter sequence 450. In certain embodiments, the primer sequence 440 is a T7-polyA primer, and the promoter sequence 450 is a T7 promoter recognized by T7 RNA polymerase, which can be utilized downstream to drive vitro T7 transcription for achieving linear amplification of the genomic DNA 208. However, other linear primers, promoters, and/or linear amplification mediators are also contemplated, such as mediators for strand displacement or PCR-type amplification. In certain embodiments, the transposon 420 includes one or more primer and/or promoter sequences recognized by T3 RNA polymerase, SP6 RNA polymerase, or DNA polymerase.

Indexing at operation 106 is achieved by the inclusion of at least one barcode sequence 460 in the transposon 420 for tagmentation of genomic DNA 208, thus not requiring any indexing ligation steps. Avoiding ligation-based indexing prevents inefficiencies in indexing, and further increases genomic coverage and uniformity of results.

Generally, barcode sequences 460 are nucleic acid sequence tags that are attached to target nucleic acids, e.g., the genomic DNA 208, for purposes of identifying or tracking the identity of individual single cells, e.g., cells 204, across one or more genome editing events. Barcode sequences 460 can be any suitable number of nucleotides in length, such as 1, 2, 3, 4, 5, 6, 7, 8, 9, 10, 11, 12, 13, 14, 15, 16, 17, 18, 19, 20, or more nucleotides in length. In order to account for sequencing errors caused by alterations to the barcode sequences 460 during downstream events, the barcode sequences 460 may be designed to be distant from each other and error-correcting. For example, the barcode sequences 460 may be Hamming-type codes or Levenshtein-type codes.

In certain embodiments, two or more of the distributed subsets of gel beads 202 are exposed to transposome complexes 400 having different barcode sequences 460, resulting in genomic DNA 208 in each of the two or more subsets being tagged with different barcode sequences 460. For example, in certain embodiments, each of the distributed subsets of gel beads 202 are exposed to a transposome complex 400 having a different barcode sequence 460. In certain embodiments, two or more of the distributed subsets of gel beads 202 are exposed to transposome complexes 400 having the same barcode sequence 460. For example, in certain embodiments, each of the distributed subsets of gel beads 202 are exposed to a transposome complex 400 having the same barcode sequence 460. In such embodiments, identification of individual cells 204 is achieved by performing a second downstream indexing event utilizing a secondary barcode sequence.

Tagmentation of the genomic DNA 208 at operation 106 facilitates fragmentation of genomic DNA into oligonucleotides ranging between about 25 nucleotides and about 4,000 nucleotides in length, such as between about 35 nucleotides and about 3,000 nucleotides in length, such as between about 100 nucleotides and about 200 nucleotides in length. However, despite tagmentation by, e.g., transposome complex 400, the genomic DNA 208 remains contained inside the gel beads 202 due to the attachment of transposase enzyme subunits 410 to the fragmented oligonucleotides. Thus, the transposase enzyme subunits 410 hold the tagmented DNA together until subsequent processing, wherein the transposase enzyme may be removed, e.g., via exposure to a detergent, heat, protease for digestion, or the like.

Generally, the amount of tagmented oligonucleotides may be adjusted by varying the amount of transposome added to the distributed subsets of gel beads 202. In other words, adjusting the ratio of transposase enzyme-to-oligonucleotide will alter the tagmentation profile of a desired sample or subset. In certain embodiments, a ratio of 1:1, 1:2, 1:4, 1:5, 1:6, 1:7, 1:8, 1:9, or 1:10 enzyme: oligo is utilized, depending on a desired tagmentation profile for downstream sequencing. In certain embodiments, the tagmentation profile may further be adjusted by varying the duration of the tagmentation reaction at operation 106. For example, in certain embodiments, the tagmentation reaction may have a reaction time of about 5, 10, 15, 20, 25, 30, 35, 40, 45, or more minutes depending on the desired tagmentation profile for downstream sequencing.

### Redistribution of Gel Beads, Gap Filling, and Linear Amplification

Returning now to FIG. 1 and FIG. 2, at operation 108, the tagmented gel beads 202 are redistributed into different subsets for the remainder of sequencing library preparation, and then gap filling and linear amplification of tagmented genomic DNA 208 is performed. Similar to the above, redistribution may include multiple distribution steps wherein a population or subpopulation of the tagmented gel beads 202 is re-pooled and re-split into new wells of one or more multi-well (e.g., 96-well or 384-well) plates or other similar format having a plurality of compartments. In certain embodiments, the number of re-splitting/re-pooling steps and/or number of subsets depends on the number of different barcode sequences to be added to the tagmented genomic DNA 208. For example, the number of re-splitting and/or re-pooling steps may be based, in part, on the desire to reduce the number of amplified gel beads 202 having the same barcode sequences inserted therein, as well as the number of cells 204 desired to be assayed. Generally, the number of tagmented gel beads 202 distributed into each subset is at least 1. In certain embodiments, the number of tagmented gel beads 202 distributed into each subset is between about 10 and about 100. Methods for re-pooling and re-splitting tagmented gel beads 202 into subsets are known to those of ordinary skill in the art and are routine.

The tagmented genomic DNA 208, now redistributed into new wells, is exposed to a transposase removal process followed by gap filling. As described above, tagmentation with a transposition system results in transposase enzyme subunits, e.g., Tn5, attaching to and holding together tagmented genomic DNA. In order to remove the transposase enzyme and separate the tagmented genomic DNA 208, gel beads 202 may be chemically treated with a detergent or other reagent capable of disrupting nucleic acid-protein interactions. Examples of suitable detergents include, but are not limited to, sodium dodecyl sulphate (SDS), which can denature secondary and non-disulfide-linked tertiary protein structures without disrupting nucleic acid-nucleic acid interactions. Upon removal of the transposase, the tagmented and fragmented genomic DNA 208, due to its small size, may diffuse out of the gel beads 202 through pores formed therein.

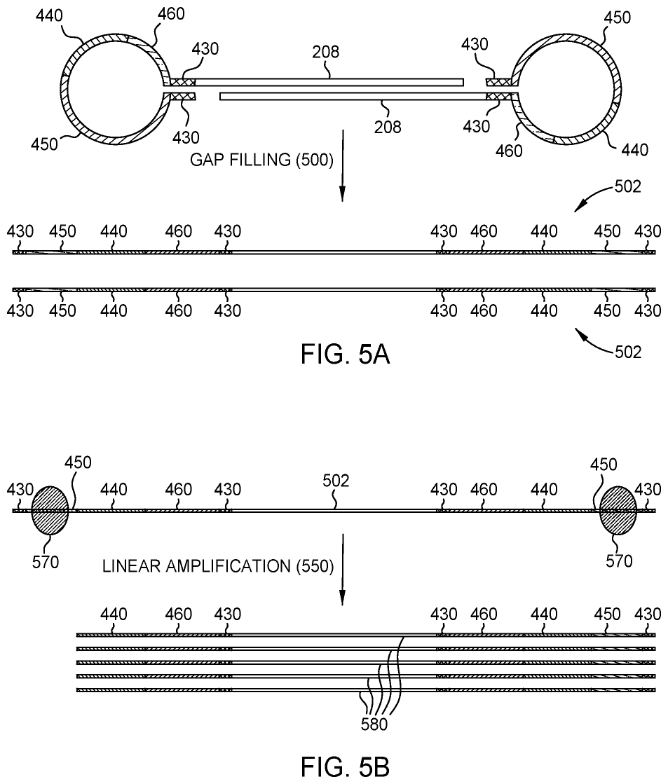

Gap filling, otherwise known as gap extension, fills single stranded gaps created in the tagmented genomic DNA 208 and converts the looped promoter sequence 450 into a duplex promoter. An exemplary gap filling reaction 500 is schematically depicted in FIG. 5A for reference. In examples wherein a promoter sequence 450 is disposed on both ends of a fragment of tagmented genomic DNA 208, such as in FIG. 5A, gap filling results in an extension product 502 having a symmetrical duplex structure. Typically, gap filling is achieved utilizing a polymerase with strand displacement activity. Examples of suitable polymerases include, but are not limited to, Bst polymerase. In certain embodiments, the gap filling reaction 500 includes the addition of one or more reagents to recover downstream polymerase activity that may be lost upon exposure of the tagmented genomic DNA 208 to the transposase removal process. For example, in certain embodiments, the gap filling reaction 500 may include the addition of a surfactant such as Tween-20 at varying concentrations, which may recover the activity of multiple types of polymerases in downstream operations, such as Q5 HiFi DNA polymerase.

In certain embodiments, after gap filling, the extension products 502 are purified using routine methods known to those of ordinary skill in the art to remove any unwanted residual reagents therefrom, such as SDS, to improve subsequent amplification efficiency and minimize amplification reaction volume. Any suitable purification or clean-up process may be used, including gel, column, or bead-based methods. Examples of suitable bead-based processes include the utilization of solid phase reversible immobilization paramagnetic beads to separate desired DNA molecules form unwanted materials.

At this stage, the double stranded extension products 502 including the tagmented genomic DNA 208 can be amplified in their respective subsets using methods known to those of ordinary skill in the art to produce amplicons of the genomic DNA 208. In certain embodiments, the amplification is a linear amplification reaction. Examples of suitable linear amplification methods include, but are not limited to, in vitro transcription (IVT), linked linear amplification, DNA polymerase-based linear amplification using a primer and thermal cycling, and other PCR-based methods. In certain embodiments, the amplification is a targeted and non-linear approach. Examples of suitable non-linear amplification methods include, but are not limited to, multiplex PCR with specific primers, loop-mediated isothermal amplification (LAMP) with specific primers, strand displacement amplification, rolling circle amplification, and ligase chain reaction.

An exemplary linear amplification reaction 550 is schematically depicted in FIG. 5B for reference. As shown in FIG. 5B, in certain embodiments, linear amplification is achieved utilizing, e.g., T7 promoter sequences 450 to drive in vitro transcription with a corresponding T7 RNA polymerase 570, which produces single stranded RNA transcripts 580 from the extension products 502. The RNA transcripts 580 produced by the reaction 550 cannot be utilized as a template for further amplification and thus, all amplified RNA copies are derived directly from the original DNA template, e.g., extension products 502. Accordingly, linear amplification methods, such as the reaction 550 depicted in FIG. 5B, may avoid several limitations of exponential amplification methods, including exponential amplification biases that skew process uniformity. Reverse Transcription, Second Strand Synthesis, and Secondary Barcode Addition

At operation 110 depicted in FIG. 1 and FIG. 2, reverse transcription of the RNA transcripts 580 is performed in respective subsets to obtain single stranded cDNA, followed by second stand synthesis to convert the single stranded cDNA into double stranded molecules. In certain embodiments, RNA transcripts 580 are purified prior to reverse transcription using routine methods known to those of ordinary skill in the art to remove any unwanted residual reagents and artifacts therefrom. Any suitable purification or clean-up process may be used for the purification process, including gel, column, or bead-based RNA extraction methods. For example, the RNA transcripts 580 may be purified using solid phase reversible immobilization paramagnetic beads.

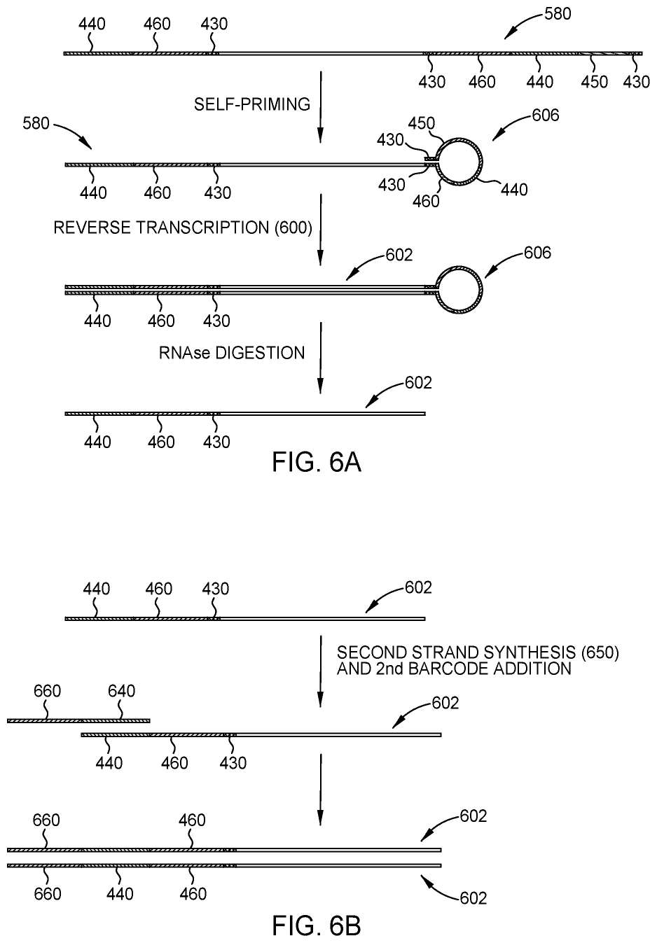

FIG. 6A schematically illustrates an exemplary reverse transcription reaction 600 for first strand synthesis, according to embodiments described herein. As shown, in certain embodiments, reverse transcription can be primed by self-priming, or self-looping, of a hairpin oligonucleotide that is inherited during tagmentation of the genomic DNA 208 at operation 106. For example, in embodiments wherein the mosaic ends 430 of the transposon 420 are complementary, the RNA transcripts 580 may be exposed to a thermocycling process to bring mosaic ends 430 together to generate a hairpin structure 606. In certain embodiments, however, additional RNA reverse transcription primers are added to the reaction 600 to drive reverse transcription of non-self-priming transcripts. For example, in embodiments where a linear oligonucleotide is inherited at operation 106, random primers or primers specifically designed against the inherited linear oligonucleotide may be utilized to drive the reverse transcription reaction 600.

In certain embodiments, the resulting cDNA-RNA duplex is treated with a ribonuclease (RNase) to digest the RNA template and purify synthesized cDNA, which is labeled as 602 in FIG. 6A. The RNase can include any of the RNases known to those of ordinary skill in the art, including but not limited to RNase H, A, T1, V1, and the like. In certain embodiments, hydrolysis of the RNA is performed.

FIG. 6B schematically illustrates an exemplary second strand synthesis reaction 650, according to embodiments described herein. Typically, a complementary second strand for cDNA 602 is synthesized via primed synthesis utilizing primers 640, which may be random primers or primers specifically designed against single stranded cDNA 602 (e.g., against oligonucleotides inherited during prior tagmentation steps). However, any suitable synthesis methods may be utilized, and in certain embodiments, second strand synthesis is performed with no priming. Examples of suitable polymerases that may be utilized for second strand synthesis include Q5 HiFi DNA polymerase and the like.

In certain embodiments, the second strand synthesis reaction 650 is carried out with user-defined primers 640 bearing secondary barcode sequences 660 that are incorporated into the second strand of cDNA 602 during synthesis. Accordingly, the second strand synthesis reaction 650 results in double stranded and barcoded cDNA 602. In certain embodiments, each subset of cDNA 602 is exposed to barcoded primers 640 having at least one secondary barcode sequence 660 unique to that respective subset. The combinatorial indexing with barcode sequences 460 and secondary barcode sequences 660 creates a sufficient amount of barcode combinations such that each single cell 204 is represented by a unique combination of barcode sequences 460 and secondary barcode sequences 660, thus enabling tracing of individual single cells across multiple genome editing events upon downstream sequencing steps. Similar to barcode sequences 460, secondary barcode sequences can be any suitable number of nucleotides in length, such as 1, 2, 3, 4, 5, 6, 7, 8, 9, 10, 11, 12, 13, 14, 15, 16, 17, 18, 19, 20, or more nucleotides in length

### Sequencing and Preparation

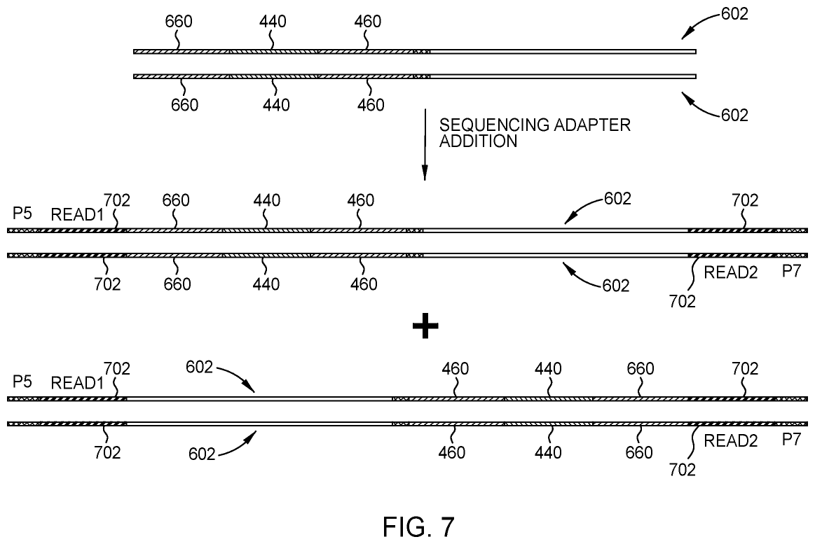

At operation 112, the double stranded cDNA 602 from all subsets (e.g., plate wells) is pooled together and prepared for sequencing. Preparation typically involves ligating sequencing adapters to the ends of the fragments of cDNA 602, schematically illustrated in FIG. 7 for reference. The sequencing adapters, labeled as 702 in FIG. 7, may be universal and identical, or varying in length and/or sequence. Ligation thereof may be achieved via blunt-ended ligation, or single-stranded overhangs may be produced via utilization of a polymerase such as Taq polymerase or Exo-Minus Klenow polymerase. In certain embodiments, the sequencing adapters 702 include all features necessary for subsequent sequencing operations, such as sequencing primer binding sites and/or anchor sequences for immobilizing the cDNA 602 on a flow cell array, among other features. In certain other embodiments, the sequencing adapters 702 include only a portion of features necessary for sequencing, and thus may require further modification via, e.g., PCR-based methods prior to sequencing.

Upon ligation of sequencing adapters 702, the resulting fragments of cDNA 602 collectively provide a library of nucleic acids for sequencing, e.g., a sequencing library, which can be utilized to trace genomic edits back to individual cells by identifying unique single cell barcode combinations (e.g., first and secondary barcode combinations). Sequencing may be carried out via any suitable sequencing techniques and platforms, including but not limited to sequencing by synthesis (SBS) on an Illumina platform (Illumina, San Diego, Calif.), single-molecule real-time sequencing (SMRT) on a Pacific Biosciences platform (Pacific Biosciences, Menlo Park, Calif.), ion semiconductor sequencing on an Ion Torrent platform (Ion Torrent Systems, Inc., Gilford, N.H.) or 454 Life Sciences platform (454 Life Sciences, Branford, Conn.), pyrosequencing, combinatorial probe anchor synthesis (cPAS) on a BGI platform (BGI Group, Shenzhen, China), sequencing by ligation, nanopore sequencing on an Oxford Nanopore platform (Oxford Nanopore Technologies Limited, Oxford, UK) and other third-generation sequencing techniques or platforms.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Other equivalent methods, steps and compositions are intended to be included in the scope of the invention. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric.

### Example 1: Encapsulating Cells in Hydrogel Beads for Compartmentalization of Single Cells

### Tris-Buffered Saline-EDTA-Triton (TB SET) Buffer:

˜1 L of TB SET buffer was prepared by combining 10 mL of 1 M Tris-HCl (pH 8.0) (final concentration: 10 mM), 137 mL of 1 M NaCl (final concentration: 137 mM), 1.35 mL of 2 M KCl (final concentration: 1.35 mM), 20 mL of 0.5 M EDTA (pH 8.0) (final concentration: 10 mM), and 10 mL of 10% (wt/vol) Triton X-100 (final concentration: 0.10%) in 822 mL of nuclease-free water and stored at room temperature.

### 10% Ammonium Persulfate (APS):

1 mL of 10% (wt/vol) APS was prepared by combining 100 mg of ≥98% (wt/vol) APS (Sigma-Aldrich, St. Louis, Mo,) in up to 1 mL of nuclease-free water and stored at −20° C. as aliquots.

### 40% Acrylamide/N,N′-Bis(Acryloyl)Systamine (BAC) Solution 20:1:

˜10 mL of 40% (wt/vol) acrylamide/BAC solution 20:1 was prepared by combining 10 mL of ˜40% (wt/vol) acrylamide (Sigma-Aldrich, St. Louis, Mo.) and 0.2 g of BAC (Sigma-Aldrich, St. Louis, Mo.) and incubating in a 60° C. bath for 1 hour to completely dissolve the BAC.

Cell Suspensions (All Suspensions were Adjusted to a Final Concentration of 1×10⁴−1×10⁵ cells per μL):

Bacterial: E. coli cells were washed and resuspended in phosphate-buffered saline (PBS), and then counted using an Accuri C6 Plus cytometer (BD Biosciences, Franklin Lakes, N.J.).

Yeast: Yeast cells grow in culture as clumps and singulating them can be challenging. Spheroplasting breaks the yeast cell wall and facilitates singulation of individual yeast cells. Therefore, yeast cells were used in the presently described protocol after spheroplasting to ensure single cell resolution.

S288c and Cen.PK yeast cells were spheroplasted in spheroplasting buffer (50 mM Phosphate buffer (pH 7.5), 1M Sorbitol, 10 mM EDTA, 2 mM DTT, 400 μg/mL nuclease and DNA/RNA free bovine serum albumin (BSA), 0.05 U/μL zymolase enzyme) and were washed and resuspended in a solution of 10 mM Tris-HCl (pH 8.0) and 1 M Sorbitol, and then counted using the Accuri C6 Plus cytometer (BD Biosciences, Franklin Lakes, N.J.).

Mammalian cells: HEK293 cells were washed and resuspended in PBS, and then counted using a Countess II Automated Cell Counter (Thermo Fisher Scientific, Waltham, Mass.).

### 24% Acrylamide-BAC (AB) Solution:

Acrylamide-BAC was used as the polymer solution as it allows dissolution of the polymer network on demand upon exposure to reducing agents such as DTT.

1 mL of 24% (wt/vol) AB solution was prepared by combining 258 μL of ˜40% (wt/vol) acrylamide and 360 μL of 40% (wt/vol) acrylamide/BAC solution 20:1 in 382 μL of nuclease-free water.

### Oil Phase:

303 μL of oil phase for water-oil emulsion formation was prepared by combining 300 μL of QX200™ Droplet Generator oil (Bio-Rad, Hercules, Calif.) and 3 μL of N, N, N′, N′-Tetramethylethylenediamine (TEMED) (Sigma-Aldrich, St. Louis, Mo.) in a first 1.5 mL microcentrifuge tube (Eppendorf, Hamburg, Germany).

### Hydrogel Polymer:

200 μL of hydrogel polymer was prepared in a second 1.5 mL microcentrifuge tube by combining 80 μL of 24% (wt/vol) AB solution, 20 μL of TB SET buffer, 10 μL of 1 M Tris-HCl (pH 8.0), cell suspension having 1×10⁶ cells total, and 6 μL of 10% (wt/vol) APS in up to 84 μL of nuclease-free water. 10% (wt/vol) APS was added to the polymer solution just prior to mixing with the oil phase.

### Cell Encapsulation Protocol:

˜200 μL of hydrogel polymer solution was dispensed over the oil phase in the first microcentrifuge tube and the tube thereafter vortexed for 25 s at ˜3,000 rpm to form an emulsion having single cells trapped within individual droplets. The emulsion was further mixed via repetitive pipetting using a 1 mL air displacement micropipette set to 600 uL, and then passed through a 40 μm Flowmi™ cell membrane (SP Bel-Art Scienceware, Wayne, N.J.) into a new 1.5 mL microcentrifuge tube to adjust droplet size. The emulsion was thereafter incubated for at least 4 hours at room temperature to polymerize droplets into hydrogel beads for encapsulation of the cells.

The number of singly-loaded hydrogel beads was controlled by varying cell concentration of the hydrogel polymer solution. Cell encapsulation in hydrogel beads followed a Poisson distribution and thus, adjusting the concentration of cells in the hydrogel polymer solution enabled tuning of the fraction of hydrogel bead loaded with single cells. For example, a concentration of 4 cell/nL resulted in 5% of hydrogel beads being loaded with single cells; a concentration of 8 cell/nL resulted in 9% of hydrogel beads being loaded with single cells; and a concentration of 20 cell/nL resulted in 15% of hydrogel beads being loaded with single cells. Most hydrogel beads during cell encapsulation were left empty to prevent encapsulating more than one cell per bead, thus ensuring single cell resolution.

### Example 2: Cell Lysis in Hydrogel Beads for Extraction of Genomic DNA

### 20% Perfluorooctanol (PFO) Solution:

1 mL of 24% (vol/vol) PFO solution was prepared by combining 200 μL of 100% (vol/vol) PFO (Sigma-Aldrich, St. Louis, Mo.) with 800 μL of HFE-7500 (Oakwood Chemical, West Columbia, S.C.).

### Cell Lysis Buffer:

65 μL of cell lysis buffer was prepared by combining 10 μL of 1 M Tris-HCl (pH 8.0) (final concentration: 10 mM), 2 μL of 0.5 M EDTA (pH 8.0) (final concentration: 1 mM), and 10% (wt/vol) SDS solution (Sigma-Aldrich, St. Louis, Mo.) (final concentration 0.50%).

### Cell Lysis Protocol:

Oil from the previously-incubated hydrogel bead solution was removed from the bottom of the microcentrifuge tube and 500 μL of TB SET buffer and 1 mL of 20% (vol/vol) PFO solution was added therein. The microcentrifuge tube was vortexed for 5 s at ˜3,000 rpm to break apart the emulsion and thereafter centrifuged at 5,000 g for 30 s to separate the contents therein. PFO was removed from the bottom of the microcentrifuge tube and 1 mL of TBSET was mixed in by repetitive pipetting. Upon the remainder of the PFO settling, ˜900 μL of the hydrogel bead solution was removed from a top portion of the microcentrifuge tube and transferred into a new 1.5 mL microcentrifuge tube. The hydrogel beads were then washed with 1 mL of PBS at least three times. To hydrolyze cell walls in E. coli cells (this step was omitted for yeast and mammalian cells), the hydrogel beads were resuspended in 1 mg/mL lysozyme solution (Thermo Fisher Scientific, Waltham, Mass.) in 1 mL PBS, incubated for 20 m at room temperature, and washed with 1 mL of PBS at least two times.

After washing, the hydrogel beads were resuspended and mixed with 1 mL of cell lysis buffer to lyse the cells and extract genomic DNA therefrom, followed by centrifuging for 30 s at 5,000 g. A ˜400 mL aliquot was removed from the suspension, leaving ˜600 μL remaining in the microcentrifuge tube. The remaining hydrogel bead suspension was mixed via repetitive pipetting and 75 μL aliquots were transferred into PCR strip tubes for thermocycling (C1000 Thermal Cycler, Bio-Rad, Hercules, Calif.) to facilitate lysis. The thermocycler was run utilizing the following program parameters:

Lid temperature: 85° C.

Reaction volume: 75 μL

| Step | Temperature (° C.) | Time |
|---|---|---|
| Incubate | 72 | 10 m |
| Hold | 4 | Infinite |

After thermocycling, the hydrogel beads were transferred to a single 1.5 mL microcentrifuge tube, wherein the combined hydrogel bead suspension had a total volume of 600 μL. To further digest cellular proteins and inactivate any nucleases, proteinase K (Thermo Fisher Scientific, Waltham, Mass.) was added to the hydrogel beads in a quantity yielding a final concentration of 400 μg/mL, and the hydrogel beads were thereafter incubated at room temperature for 15 m. The hydrogel beads were then washed with 1 mL PBS at least three times and resuspended in PBS at a final volume of 450 mL. 450 μL of 0.4 M NaOH was added to the hydrogel bead suspension and mixed, and the suspension was incubated at room temperature for 5 m. 300 μL of 1 M Tris-HCl (pH 7.2) was added and mixed with the suspension to neutralize the NaOH solution before the hydrogel beads were washed once with 10 mM Tris-HCl buffer (pH 8.0). The hydrogel beads were then resuspended in 1 mL of 10 mM Tris-HCl (pH 8.0) and stored at −80° C. for up to a month.

Note that the genomic DNA from lysed cells remained inside the hydrogel beads, even after cell lysis and proteinase digestion, due the large size of the DNA and branching thereof.

### Example 3: Whole Genome Amplification in Hydrogel Beads to Increase Volume of Genomic Material for Downstream Events

Once the cells were lysed and genomic DNA extracted therefrom, the genomic DNA was amplified within the hydrogel beads in order to increase the amount of single cell genomic material available for library preparation. Amplification was performed utilizing the QIAGEN REPLI-g Single Cell Kit (QIAGEN, Hilden, Germany). First, suspended hydrogel beads were centrifuged for 30 s at 5,000 g, and the supernatant therefrom removed. Thereafter, 10 μL of hydrogel beads, 9 μL of nuclease-free water, 29 μL of REPLI-g buffer, and 2 μL of REPLI-g phi29 enzyme were added, respectively, to a new 150 mL microcentrifuge tube and temperature cycled to facilitate the amplification reaction with the following parameters:

Lid temperature: 70° C.

Reaction volume: 50 μL

| Step | Temperature (° C.) | Time |
|---|---|---|
| Incubate | 30 | 30 m |
| Inactivate | 65 | 5 m |
| Hold | 4 | Infinite |

The hydrogel beads were transferred to a new 150 mL microcentrifuge tube, washed with 1 mL Low TE buffer (10mM Tris-HCl (pH 8.0), 0.1 mM EDTA) at least twice, and resuspended in 1 mL Low TE buffer. For quality control, 20 μL of the hydrogel bead suspension was removed and stained for fluorescence imaging. The hydrogel beads were stained with SYTOX® Green fluorescent stain (Thermo Fisher Scientific, Waltham, Mass.) and imaged with a fluorescence microscope to determine the number of beads loaded with single cells, and this number was utilized to estimate the concentration of cells in the hydrogel bead suspension. The remaining amplified hydrogel beads were stored at −80° C. for up to a month.

### Example 4: First Level Barcoding of Amplified DNA via Tagmentation

### 10X Annealing Buffer:

10X annealing buffer was prepared by combining 10 μL of 1 M Tris-HCl (pH 7.5) per 1 mL buffer (final concentration: 10 mM), 100 μL of 5 M NaCl per 1 mL buffer (final concentration: 500 mM), 20 μL of 0.5 M EDTA per 1 mL buffer (final concentration: 10 mM), and 870 μL nuclease-free water per 1 mL buffer.

### Tn5 Dilution Buffer:

Tn5 dilution buffer was prepared by combining 5 mL of 100% (vol/vol) glycerol per 10 mL buffer (final concentration: 50%), 500 μL of 1 M Tris-HCl (pH 7.5) per 10 mL buffer (final concentration: 50 mM), 200 μL of 5 M NaCl per 10 mL buffer (final concentration: 100 mM), 2 μL of 0.5 M EDTA per 10 mL buffer (final concentration: 0.1 mM), 10 μL of 100% (wt/vol) Triton X-100 per 10 mL buffer (final concentration: 0.10%), and 4,288 μL nuclease-free water per 10 mL buffer.

### Assembling Barcoded Transposomes:

100 μM custom barcoded transposon oligonucleotide stocks were ordered and commercially synthesized by Integrated DNA Technologies, Inc., Coralville, Iowa, for purposes of combinatorial barcode tracking of editing events to the genomic DNA. The barcoded transposon stocks included transposons comprised of either hairpin oligonucleotides, thus enabling self-priming during downstream reverse transcription, or linear oligonucleotides, requiring external priming during downstream events. The barcoded transposons were annealed by mixing the following reagents in each reaction well of a 96-well plate for each individual barcoded transposon (e.g., one well of the 96-well plate per unique barcode, 96 unique barcodes per one 96-well plate): 5 μL of 10X annealing buffer (final concentration: 1X); 22.5 μL of 100 μM custom (e.g., user defined) transposon (final concentration: 45 μM); and 22.5 μL of nuclease-free water. Annealing was facilitated by temperature cycling with the following parameters (in sequence):

95° C. for 3 m;

70° C. for 3 m;

Ramp down to 25° C. at 2 C/m.

After thermocycling, 180 μL of nuclease-free water was added to each reaction well having annealed transposon therein. 2.5 μL of the diluted transposon was mixed with 2.5 μL of 100% (vol/vol) glycerol, which was heated to 60° C. and then cooled to room temperature prior to mixing. 5 μL of 1 U/μL Tn5 transposase (Lucigen, Middleton, Wis.) was then mixed into the diluted transposon to form loaded transposome complexes and the mixture was incubated for 30 m at 25° C. in a thermocycler with lid temperature set to 30° C. At this point, the loaded Tn5 transposome was stored at −20° C. until further dilution with Tn5 dilution buffer. Dilution of the loaded Tn5 transposome included a 11.25-fold dilution for each reaction well. For example, for a total volume of 22.5 μL in each reaction well, 2 μL of loaded Tn5 transposome in each well was diluted with 20.5 μL of Tn5 dilution buffer. The diluted transposome was then stored again at −20° C. for subsequent tagmentation operations.

### Tagmentation Stop Solution:

Tagmentation stop solution was prepared by combining 24.75 μL of 100 mM spermidine (Sigma-Aldrich, St. Louis, Mo.) per 1.5 mL solution (final concentration: 1.65 mM) and 997.5 μL of 100 mM EDTA per 1.5 mL solution (final concentration: 66.5 mM) in nuclease free water.

### Tagmentation Protocol:

To fragment and tag (e.g., index) extracted DNA in the hydrogel beads, 96 unique tagmentation reactions were prepared (e.g., 1 unique reaction/well) by mixing the following reagents in each reaction well of the 96-well plate: 10 μL of Nextera® XT Tagment DNA (TD) buffer (Illumina, San Diego, Calif.), hydrogel beads containing a total of 1,000-1,500 cells, 4 μL of loaded Tn5 transposome, and up to 20 μL of nuclease-free water. The cell-loaded hydrogel bead concentration (loaded cells/μL) determined in Example 3 was utilized to ensure 1,000-1,500 cells were loaded into each reaction well. Loaded wells were incubated for 10 m at 55° C. in a thermocycler having the lid set to 105° C., and thereafter held at 10° C. The samples were immediately removed and 5 μL of tagmentation stop solution was added into each reaction well. The plates were subsequently spun down and incubated with the tagmentation stop solution on ice for 5 m.

Downstream in vitro transcription products for both hairpin and linear transposon designs showed fragment profiles (e.g., number and/or size of tagmented DNA fragments) ranging from 100 to 2,000 bases in length as a result of the tagmentation protocol above. Upon performance of multiple experiments, it was determined that fragmentation profile could be adjusted by varying the amount of loaded transposome added to the reaction mix and/or changing the length of incubation. Thus, a desired fragmentation profile could be achieved by changing the amount of added transposome and/or changing the length of incubation.

### Example 5: Redistribution of Tagmented Hydrogel Beads

Protocol for Pooling and Splitting Tagmented Hydrogel Beads into a New Multi-Well Plate:

Following tagmentation, the contents (about 25 μL) from each reaction well were combined into a 10 mL trough via multichannel air displacement pipetting and transferred into a 50 mL centrifuge tube. The combined solution was mixed by inversion and centrifuged to pellet the suspended hydrogel beads. The supernatant was thereafter removed and the pellet resuspended in 16 mL of fresh 10 mM Tris-HCl (pH 8.0), resulting in a concentration of 6 hydrogel beads per μL. 2 μL of the resuspended hydrogel bead solution was then pipetted into each reaction well of a new 96-well plate such that each well was allocated 12 cells.

### Example 6: Tn5 Transposase Removal and Gap Extension

### Gap Extension Master Mix:

Gap extension master mix was prepared by combining 10 μL of Q5® High Fidelity 2X Master Mix (New England Biolabs, Ipswich, Mass.) per 17 μL master mix (final concentration: 1X), 2 μL of 100 mM dithiothreitol (DTT) (Invitrogen, Carlsbad, Calif.) per 17 μL master mix (final concentration: 10 mM), 3 μL of 10% (wt/vol) Tween-20 (Bio-Rad, Hercules, Calif.) per 17 μL master mix (final concentration: 1%), and 3 μL of nuclease-free water per 17 μL master mix.

### Protocol for Tn5 Removal and Gap Extension:

Even after tagmentation, genomic DNA remained inside the hydrogel beads due to the binding of Tn5 transposase enzyme to tagmented DNA fragments. To facilitate diffusion of the genomic DNA out of the hydrogel beads, Tn5 was washed out from each of the newly redistributed wells with detergent. 1 μL of 0.12% (wt/vol) SDS solution (Promega Corporation, Madison, Wis.) was added to each reaction well and mixed via repetitive pipetting. The samples were spun down and incubated at room temperature for 5 m. 17 μL of the gap extension master mix was then allocated to each reaction well, and the samples were incubated for 10 m at 72° C. in a thermocycler with the lid set to 105° C. After holding the samples at 4° C., the samples were removed from the thermocycler and purified via a solution exchange. Note that Tween-20 was included in the master mix in order to facilitate Tn5 removal by SDS, which kills downstream activity of polymerases. By adding Tween-20, downstream polymerase activity was rescued.

### Example 7: Sample Preparation for Linear Amplification

### DNA Purification Protocol:

Tagmented DNA was purified and concentrated using AMPure® XP Beads (Beckman Coulter Life Science, Indianapolis, Ind.) in order to improve subsequent amplification efficiency and minimize amplification reaction volume. AMPure® XP Beads were first equilibrated to room temperature and distributed in 40 μL volumes to each reaction well having tagmented DNA therein. The multi-well plate was shaken until the samples were homogenous and then incubated at room temperature for 5 minutes, after which the plate was spun down and placed on a magnetic rack. Upon formation of pellets in each reaction well (about 2 m), 190 μL of supernatant was removed and exchanged with 190 μL of 80% (vol/vol) ethanol without disturbing the pellets, thus cleaning the samples of unwanted reagents and/or artifacts. The multi-well plate was incubated for 30 minutes, and the solvent exchange and incubation were thereafter repeated for at least two more washes. After washing, the remaining ethanol in each reaction well was removed, and tagmented DNA was eluted from the hydogel beads by adding 9 μL of Tris-HCl to each well. Samples were shaken to resuspend the hydrogel beads and then incubated for 5 m, spun down, and repositioned on the magnetic rack. 8 μL of eluted DNA from each reaction well was transferred into a new well of a multi-well plate for subsequent amplification.

### Example 8: Linear Amplification of Tagmented DNA Fragments for Library Preparation

### Linear Amplification Protocol:

Tagmented DNA was linearly amplified via T7 in vitro transcription (IVT) using the HiScribe™ T7 Quick High Yield RNA Synthesis Kit (New England Biolabs, Ipswich, Mass.) in order to improve accuracy and uniformity in downstream sequencing data, as compared to exponential amplification methods. 12 μL of IVT Master Mix from the HiScribe™ kit was mixed with the 8 μL of eluted DNA in each well. The IVT Master Mix included 2 μL of 10X IVT buffer per 12 μL of Master Mix (final concentration: 1X), 2 μL of 100 mM ATP per 12 μL of Master Mix (final concentration: 10 mM), 2 μL of 100 mM CTP per 12 μL of Master Mix (final concentration: 10 mM), 2 μL of 100 mM GTP per 12 μL of Master Mix (final concentration: 10 mM), 2 μL of 100 mM GTP buffer per 12 μL of Master Mix (final concentration: 10 mM), and 2 μL of 10X T7 RNA Polymerase per 12 μL of Master Mix (final concentration: 1X). The samples were then incubated in a thermocycler for 16 h at 37° C., followed by an infinite hold at 4° C.

### Example 9: Post-Amplification Sample Purification

### RNA Transcript Purification Protocol:

The RNA transcripts obtained from IVT were purified and concentrated using RNAClean XP Beads (Beckman Coulter Life Science, Indianapolis, Ind.). RNAClean XP Beads were first equilibrated to room temperature and distributed in 40 μL volumes to each reaction well having RNA transcripts therein. The multi-well plate was shaken until the samples were homogenous and then incubated at room temperature for 5 minutes, after which the plate was spun down and placed on a magnetic rack. Upon formation of pellets in each reaction well (about 2 m), 200 μL of supernatant was removed and exchanged with 190 μL of 80% (vol/vol) ethanol without disturbing the pellets, thus cleaning the samples of unwanted reagents and/or artifacts. The multi-well plate was incubated for 30 minutes, and the solvent exchange and incubation were thereafter repeated for at least two more washes. After washing, the remaining ethanol in each reaction well was removed, and the RNA transcripts were eluted from the hydogel beads by adding 20 μL of Tris-HCl to each well. Samples were shaken to resuspend the hydrogel beads and then incubated for 5 m, spun down, and repositioned on the magnetic rack. 16 μL of eluted RNA transcripts from each reaction well was transferred into a new well of a multi-well plate for further processing.

### Example 10: Post-Amplification RNA Integrity Assessment

### Analysis of RNA via Electrophoresis:

A quality check was performed on the resulting RNA transcripts using the High Sensitivity RNA ScreenTape System (Agilent Technologies, Santa Clara, Calif.). Samples for the electrophoresis assay were prepared by combining 1 μL of High Sensitivity Sample Buffer and 2 μL of solution from a single well of amplified RNA transcripts into a microcentrifuge tube for each sample lane. A ladder lane was also prepared with 2 μL of High Sensitivity RNA Ladder and 1 μL of High Sensitivity Sample Buffer. Samples were spun down, vortexed for 1 m at 2,000 rpm, and then spun down again prior to incubation at 72° C. for 3 minutes and placement on ice for 2 m. The samples were then run on the 2200 TapeStation system, wherein each sample lane corresponded to a single reaction well of RNA transcripts from the amplification reaction. The resulting RNA profile included fragments ranging from about 100 bases to about 1,000 bases, with a concentration of about 1 ng/μL or more, thus confirming the integrity of the RNA transcripts.

### Example 11: Reverse Transcription, Second Strand cDNA Synthesis, and Second Level Barcoding to Facilitate Combinatorial Barcode Tracking of Single Cells

### Denaturation/Self-Priming Master Mix:

Denaturation and self-priming master mix was prepared by combining 2 μL of 10 mM dNTPs (New England Biolabs, Ipswich, Mass.) per 3.6 μL of master mix (final concentration: 0.666 mM), 1 μL of 20 U/μL SUPERase RNAse inhibitor (Invitrogen, Carlsbad, Calif.) per 3.6 μL of master mix (final concentration: 0.667 U/μL), and 0.6 μL of nuclease-free water per 3.6 μL of master mix.

### Reverse Transcription Master Mix:

Reverse transcription master mix was prepared by combining 6 μL of 5X SuperScript IV Buffer (Invitrogen, Carlsbad, Calif.) per 9.5 μL of master mix (final concentration: 1X), 1.5 μL of 100 mM DTT per 9.5 μL of master mix (final concentration: 5 mM), 1 μL of 20 U/μL SUPERase RNAse inhibitor per 9.5 μL of master mix (final concentration: 0.667 U/μL), and 1 μL of SuperScript IV Reverse Transcriptase (Invitrogen, Carlsbad, Calif.) per 9.5 μL of master mix (final concentration: 6.67 U/μL).

### RNAse Master Mix:

RNAse master mix was prepared by combining 0.5 μL of 5,000 U/mL RNAse H (New England Biolabs, Ipswich, Mass.) per 0.8 μL of master mix (final concentration: 83.33 U/mL) and 0.3 μL of 1 mg/mL RNAse A (Invitrogen, Carlsbad, Calif.) per 0.8 μL of master mix (final concentration: 0.01 mg/mL).

cDNA Second Strand Synthesis Master Mix:

cDNA synthesis master mix was prepared by combining 10 μL of 5X Q5 High GC Enhancer (New England Biolabs, Ipswich, Mass.) per 21.5 μL of master mix (final concentration: 1X), 10 μL of 5X Q5 Buffer (New England Biolabs, Ipswich, Mass.) per 21.5 μL of master mix (final concentration: 1X), 1 μL of 10 mM dNTPs (New England Biolabs, Ipswich, Mass.) per 21.5 μL of master mix (final concentration: 0.2 mM), and 0.5 μL of 20 U/μL Q5 High Fidelity DNA Polymerase (New England Biolabs, Ipswich, Mass.) per 21.5 μL of master mix (final concentration: 0.2 U/μL).

Protocol for Reverse Transcription and Second Strand Synthesis with Barcoded Primers:

Reverse transcription of the RNA transcripts was performed to form single stranded cDNA from the RNA transcripts produced during IVT, followed by second stand synthesis to convert the single stranded cDNA into double stranded molecules. For second strand synthesis, barcoded primers were utilized to introduce unique barcodes into individual sample reaction wells, which in combination with the first barcodes introduced via tagmentation, allowed for combinatorial indexing of during downstream sequencing.

First, 3.6 μL of denaturation/self-priming master mix was added to each reaction well having RNA therein, followed by incubation of the samples in a thermocycler set to a sequence of 70° C. for 1 m and 90° C. for 20. The samples were immediately transferred onto ice, and 9.5 μL of reverse transcription master mix was added to each sample reaction well. Another incubation in the thermocyler was then performed to facilitate the reverse transcription reaction with the following parameters (in sequence):

55° C. for 15 m;

60° C. for 10 m;

65° C. for 12 m;

70° C. for 8 m;

75° C. for 5 m;

80° C. for 10 m;

22° C. for an infinite hold.

After the second round of thermal cycling, 0.8 μL of RNAse master mix was mixed into each sample reaction well, and the samples were again incubated at 37° C. for 30 m and then held at 4° C. 0.5 μL of 1 μM barcoded primer was added to each reaction well such that each reaction well had a unique barcode, followed by 21.5 μL of cDNA synthesis master mix for second strand synthesis. The samples were then temperature cycled with the following parameters (in sequence) to facilitate synthesis:

98° C. for 40 s;

58° C. for 30 s;

60° C. for 30 s;

65° C. for 30 s;

70° C. for 30 s;

72° C. for 6 m;

4° C. for an infinite hold.

### Example 12: cDNA Purification and Pooling for Downstream Sequencing

cDNA Purification Protocol:

The double stranded cDNA formed from reverse transcription and second strand synthesis was purified and concentrated using AMPure XP Beads (Beckman Coulter Life Science, Indianapolis, Ind.). AMPure XP Beads were first equilibrated to room temperature and then distributed in 90 μL aliquots to each sample reaction well. The multi-well plate was shaken until the samples were homogenous and then incubated at room temperature for 5 minutes, after which the plate was spun down and placed on a magnetic rack. Upon formation of pellets in each reaction well (about 2 m), 200 μL of supernatant was removed and exchanged with 190 μL of 80% (vol/vol) ethanol without disturbing the pellets, thus cleaning the samples of unwanted reagents and/or artifacts. The multi-well plate was incubated for 30 minutes, and solvent exchange and incubation were thereafter repeated for at least two more washes. After washing, the remaining ethanol in each reaction well was removed, and cDNA were eluted from the hydogel beads by adding 27.5 μL of Tris-HCl to each well. Samples were shaken to resuspend the hydrogel beads and then incubated for 5 m, spun down, and repositioned on the magnetic rack. 25 μL of eluted cDNA from each reaction well was transferred into a new well of a multi-well plate for sequencing.

Prior to sequencing, purified cDNA libraries were quantified using the Quantifluor ONE® dsDNA System from Promega Corporation, Madison, Wis. Upon determining the cDNA concentration of each sample reaction well in ng/μL, the sample reaction wells were normalized to the same concentration utilizing a corresponding volume of 10 mM Tris-HCl (pH 8.0). Each well was normalized such that at least 15 ng of cDNA could be removed from into a final pool of cDNA products.

### Example 13: Sequencing Library Preparation

### Sequencing Library Preparation Protocol:

A sequencing library was prepared with the NEBNext® Ultra™ II DNA Library Prep Kit for Illumina (New England Biolabs, Ipswich, Mass.), using 1 μg of pooled cDNA as a starting material. Upon ligation of sequencing adapters, cleanup of adapter-ligated cDNA, and PCR-based amplification thereof, the sample was ready for sequencing via Illumina sequencing by synthesis (SBS) technology.

While this invention is satisfied by embodiments in many different forms, as described in detail in connection with preferred embodiments of the invention, it is understood that the present disclosure is to be considered as exemplary of the principles of the invention and is not intended to limit the invention to the specific embodiments illustrated and described herein. Numerous variations may be made by persons skilled in the art without departure from the spirit of the invention. The scope of the invention will be measured by the appended claims and their equivalents. The abstract and the title are not to be construed as limiting the scope of the present invention, as their purpose is to enable the appropriate authorities, as well as the general public, to quickly determine the general nature of the invention. In the claims that follow, unless the term “means” is used, none of the features or elements recited therein should be construed as means-plus-function limitations pursuant to 35 U.S.C. § 112, ¶6.

1. A method for preparing a sequencing library comprising nucleic acids from a plurality of single cells, the method comprising:
encapsulating a plurality of single cells in gel beads, each of the single cells comprising genomic material and encapsulated in a different gel bead;
distributing the gel beads in a plurality of first subsets, each of the first subsets disposed in an isolated first compartment;
fragmenting the genomic material of the encapsulated single cells in the first subsets into nucleic acid fragments;
introducing a first barcode sequence into the nucleic acid fragments in the first subsets;
distributing the gel beads in a plurality of second subsets, each of the second subsets disposed in an isolated second compartment;
linearly amplifying the nucleic acid fragments having the first barcode sequence in the second subsets;
introducing a second barcode sequence into the amplified nucleic acid fragments in the second subsets; and
combining the amplified nucleic acid fragments from the second subsets to generate a pooled sequencing library, the first and second barcodes introduced into the nucleic acid fragments providing combinatorial indexing for tracking of genomic events to each of the single cells in the pooled sequencing library.

2. The method of claim 1, wherein the single cells are mammalian or bacterial cells.

3. The method of claim 1, wherein the gel beads are hydrogel beads comprising polyacrylamide or polyurethane.

4. The method of claim 1, wherein the genomic material is pre-amplified prior to distributing the gel beads in the plurality of first subsets.

5. The method of claim 1, wherein fragmenting the genomic material and introducing the first barcode sequence are achieved via transposase-assisted tagmentation.

6. The method of claim 5, wherein the transposase-assisted tagmentation comprises exposing the genomic material to a transposome complex, the transposome complex comprising a Tn5 transposase bound to an oligonucleotide comprising at least the first barcode sequence.

7. The method of claim 6, wherein the oligonucleotide further comprises a primer sequence and a promoter sequence for T7 in vitro transcription.

8. The method of claim 1, linearly amplifying the nucleic acid fragments comprises T7 in vitro transcription of the nucleic acid fragments.

9. The method of claim 8, further comprising:
reverse transcribing the amplified nucleic acid fragments in the second subsets; and
synthesizing a second strand for each of the reverse-transcribed nucleic acid fragments, the second strand synthesis introducing the second barcode sequence into the nucleic acid fragments.

10. A method for preparing a sequencing library comprising nucleic acids from a plurality of single cells, the method comprising:
encapsulating a plurality of single cells in gel beads, each of the single cells comprising genomic material and disposed in a different gel bead;
distributing the gel beads in a plurality of first subsets, each of the first subsets disposed in an isolated first compartment;
fragmenting the genomic material of the encapsulated single cells in the first subsets into nucleic acid fragments;
introducing a first barcode sequence into the nucleic acid fragments in the first subsets;
distributing the gel beads in a plurality of second subsets, each of the second subsets disposed in an isolated second compartment;
transcribing the nucleic acid fragments in the second subsets into RNA transcripts to linearly amplify the nucleic acid fragments;
reverse transcribing the RNA transcripts in the second subsets into single-stranded DNA fragments;
synthesizing a second strand for each of the single-stranded DNA fragments in the second subsets to form double-stranded DNA fragments, the second strand synthesis introducing a second barcode sequence into the double-stranded DNA fragments; and
combining the double-stranded DNA fragments from the second subsets to generate a pooled sequencing library, the first and second barcodes of the double-stranded DNA fragments providing combinatorial indexing for tracking of genomic events to each of the single cells in the pooled sequencing library.

11. The method of claim 10, wherein the single cells are mammalian or bacterial cells.

12. The method of claim 10, wherein the single cells are lysed and the genomic material thereof is pre-amplified prior to distributing the gel beads into the first subsets.

13. The method of claim 10, wherein fragmenting the genomic material and introducing the first barcode sequence are achieved via transposase-assisted tagmentation.

14. The method of claim 13, wherein the transposase-assisted tagmentation further introduces a primer sequence and a promoter sequence to the nucleic acid fragments for transcription.

15. The method of claim 14, wherein the first barcode sequence, the primer sequence, and the promoter sequence are a part of an oligonucleotide that is configured to form a hairpin structure upon annealing for self-priming during the reverse transcription.

16. The method of claim 14, wherein the first barcode sequence, the primer sequence, and the promoter sequence are a part of a linear oligonucleotide that requires external priming during the reverse transcription.

17. The method of claim 10, further comprising:
performing gap extension of the nucleic acid fragments upon tagmentation.

18. The method of claim 10, wherein transcribing the nucleic acid fragments comprises T7 in vitro transcription.

19. The method of claim 10, further comprising:
ligating sequencing adapters to the double-stranded DNA fragments for sequencing.

20. A method for preparing a sequencing library comprising nucleic acids from a plurality of single cells, the method comprising:
encapsulating a plurality of single cells in hydrogel beads, each of the single cells comprising genomic material and disposed in a different hydrogel bead;
hydrolyzing the encapsulated cells and pre-amplifying the genomic material via multiple displacement amplification;
distributing the hydrogel beads in a plurality of first subsets;
tagmenting the genomic material of the single cells with a Tn5 transposition system to form nucleic acid fragments and introduce a first barcode sequence into the nucleic acid fragments;
distributing the hydrogel beads in a plurality of second subsets;
transcribing the nucleic acid fragments into RNA transcripts via T7 in vitro transcription to linearly amplify the nucleic acid fragments;
reverse transcribing the RNA transcripts into single-stranded cDNA fragments;
synthesizing a second strand for each of the single-stranded cDNA fragments to form double-stranded cDNA fragments and introduce a second barcode sequence into the double-stranded cDNA fragments; and
combining the double-stranded cDNA fragments from the second subsets to generate a pooled sequencing library, the first and second barcodes of the double-stranded cDNA fragments providing combinatorial indexing for tracking of genomic events to each of the single cells in the pooled sequencing library.
